(19) United States
(12) Patent Application Publication
(10) Pub. No.: US 2009/0043301 A1
(43) Pub. Date: Feb. 12, 2009

(54) MONOPOLAR ENERGY DELIVERY DEVICES AND METHODS FOR CONTROLLING CURRENT DENSITY IN TISSUE

(75) Inventors: Jerry Jarrard, Sunnyvale, CA (US); Huy Phan, Santa Clara, CA (US); Scott Epstein, Boston, MA (US)

Correspondence Address: PERKINS COIE LLP;PATENT SEA, P.O. BOX 1247, SEATTLE, WA, 98111, US

(73) Assignee: Asthmatx, Inc., Sunnyvale, CA (US)

(21) Appl. No.: 12/188,782

(22) Filed: Aug. 8, 2008

Related U.S. Application Data
(60) Provisional application No. 60/954,901, filed on Aug. 9, 2007.

(51) Int. Cl. A61B 18/18

(52) U.S. Cl. 606/41

(57) ABSTRACT

Devices, systems and methods for providing energy to treat large areas of tissue. Several embodiments of the devices control resistance or other parameters to provide substantially uniform current density along a length of tissue with reduced edge effects.

### CROSS-REFERENCE TO RELATED APPLICATION

The present application claims priority under 35 U.S.C. 119(e) to U.S. Provisional Application Ser. No. 60/954,901, filed Aug. 9, 2007, the disclosure of which is incorporated herein by reference in its entirety.

### TECHNICAL FIELD

The present application relates generally to medical treatment devices, such as monopolar devices that treat lung diseases by applying a controlled current density in tissue of airways to reduce the resistance to airflow.

### BACKGROUND

Asthma is a disease that makes it difficult to breathe and in many cases can be debilitating. Asthma is generally manifested by (i) bronchoconstriction, (ii) excessive mucus production, and/or (iii) inflammation and swelling of airways that cause widespread but variable airflow obstructions. Asthma can be a chronic disorder often characterized by persistent airway inflammation, but asthma can be further characterized by acute episodes of additional airway narrowing via contraction of hyper-responsive airway smooth muscle tissue.

Conventional pharmacological approaches for managing asthma include: (i) administering anti-inflammatories and long-acting bronchodilators for long-term control, and/or (ii) administering short-acting bronchodilators for management of acute episodes. Both of these pharmacological approaches generally require repeated use of the prescribed drugs at regular intervals throughout long periods of time. However, high doses of corticosteroid anti-inflammatory drugs can have serious side effects that require careful management, and some patients are resistant to steroid treatment even at high doses. As such, effective patient compliance with pharmacologic management and avoiding stimuli that triggers asthma are common barriers to successfully managing asthma.

Asthmatx, Inc. has developed new asthma treatments that involve applying energy to alter properties of the smooth muscle tissue or other tissue (e.g., nerves, mucus glands, epithelium, blood vessels, etc.) of airways in a lung of a patient. Several embodiments of methods and apparatus related to such treatments are disclosed in commonly-assigned U.S. Pat. Nos. 6,411,852, 6,634,363, and 7,027,869; and U.S. Published Application No. US2005/0010270, all of which are incorporated by reference herein in their entirety.

Many embodiments of the foregoing asthma treatments that apply energy to tissue of the airways use catheters that can be passed (e.g., navigated) through the tortuous passageways defined by the lung airways. FIG. 1, for example, illustrates a bronchial tree 90 in which the various bronchioles 92 decrease in size and have many branches 96 as they extend from the right and left bronchi 94. Accordingly, the treatment devices should be configured to treat airways of varying sizes as well as function properly when repeatedly deployed after navigating through the tortuous anatomy.

One challenge of delivering energy to the airways in the lung is that it may take three sessions of 30-60 minutes each to treat a substantial portion of the lungs of a patient (e.g., upper and lower lobes). The three treatment sessions are usually performed on separate days, so it is also desirable to reduce the time necessary for such treatments. One factor affecting the treatment time is the length or width of the electrodes that contact the airway tissue. Typical monopolar energy delivery devices have small, short electrodes that limit the size of the contact area to reduce or mitigate non-uniformities of the current density in the tissue at the electrode. More specifically, the difference between the current density in the tissue at an edge of an electrode and the center of the electrode increases with increasing electrode dimensions (e.g., electrode length) due in part to tissue resistivity characteristics. As shown in FIG. 1A, the current density I in the tissue proximate to the edges E of a large, long electrode is accordingly significantly higher than the current density I in the tissue at the center C of the long electrode. This is known as the “edge effect,” and it is generally undesirable because the higher current densities at the edges or ends of the long electrode (or several short electrodes axially spaced so that it effectively acts as one long conductive area) may ablate and/or otherwise affect the airway tissue in an undesirable manner.

Current monopolar electrode configurations are accordingly limited to relatively small electrodes (e.g., 3-5 mm long) that exhibit acceptable edge effect non-uniformities. Small electrodes, however, treat corresponding small regions of tissue. Accordingly, small electrodes are advanced axially along airways in a large number of short increments to treat long segments of airways throughout the lung of a patient. As a result, small electrodes require longer and/or more treatment sessions to treat a patient and may result in over/under treatment of long segments due to repeated re-positioning.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the disclosure and are not intended to limit the scope of the disclosure.

FIG. 1 is an illustration of the airways within a human lung.

FIG. 1A is a schematic chart illustrating current density in tissue along a length of a long electrode.

FIG. 2 is a schematic view illustrating a system with a power/control unit and an energy delivery device for delivering energy to tissue according to an embodiment of the technology.

FIG. 3 is a side cross-sectional view illustrating an energy applicator including an expandable member and conductive ring electrodes in accordance with an embodiment of the disclosure.

FIG. 3A is a schematic chart illustrating current density in tissue along a length of an energy applicator.

FIG. 4A is a side cross-sectional view illustrating an energy applicator including an expandable member and a conductive solution in accordance with another embodiment of the disclosure.

FIG. 4B is a side cross-sectional view illustrating yet another embodiment of an energy applicator.

FIG. 4C is an isometric view illustrating a portion of the energy applicators of FIGS. 4A and 4B in more detail.

FIG. 5 is a side cross-sectional view illustrating an additional embodiment of an energy applicator.

FIG. 6 is a side cross-sectional view illustrating still another embodiment of an energy applicator.

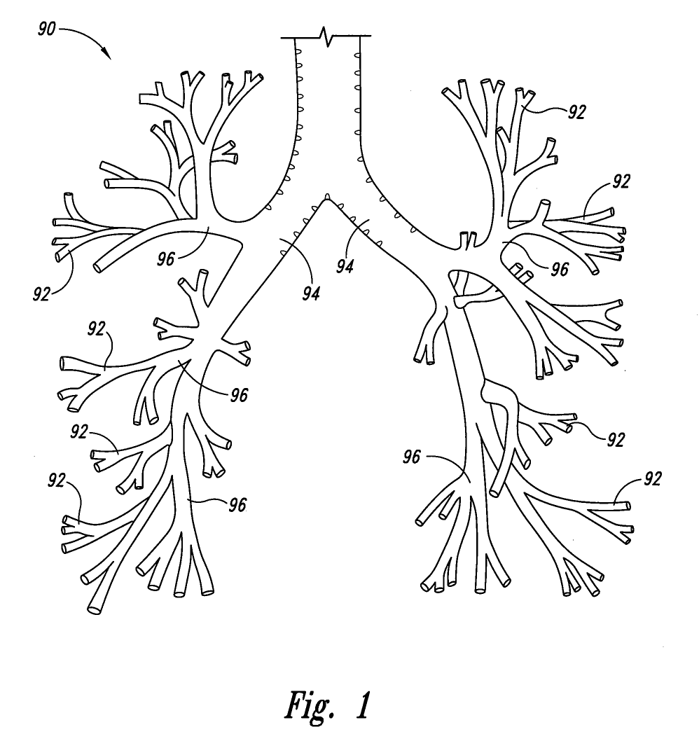

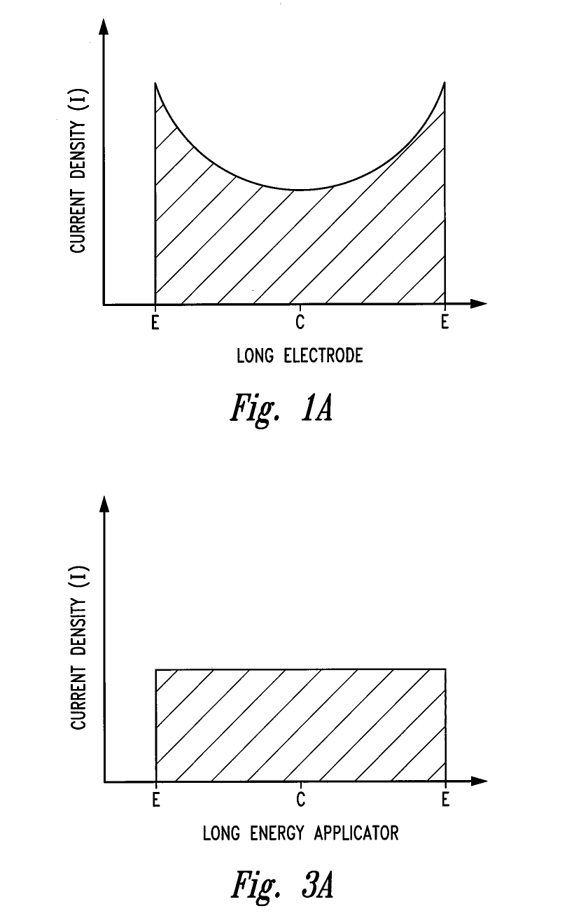

### DETAILED DESCRIPTION

### Overview

Many examples of monopolar devices, systems, and methods for controlling the delivery of energy to tissue within a body cavity are described in this section. Several examples of such systems have an energy delivery device configured to control the current density in tissue along an extended length. Thus, several embodiments of systems in accordance with the technology have relatively larger or longer conductive areas that can treat large cross-sectional areas of tissue within a given time period to reduce the number of treatment sites and cycles compared to shorter or otherwise smaller electrodes. Accordingly, shorter and/or fewer treatment sessions are needed to treat a patient and improved treatment consistency is achieved with longer conductive areas.

Specific examples of the system include an energy delivery device having an elongate member and an energy applicator at a distal portion of the elongate member. The energy applicator, for example, can include an expandable member and an energy conductor associated with the expandable member that are configured to vary the amount of energy (e.g., voltage) delivered along the length of the energy applicator in a manner that controls the current density in tissue to avoid edge effects. In certain embodiments, the expandable member is a non-conductive balloon and the energy conductor is an electrically conductive fluid. The balloon, for example, may be made from a micro-porous material through which the conductive fluid can seep or otherwise pass to contact a passageway wall. The pores can have varying sizes (e.g., diameters), shapes, number, thickness, spacing, densities, or physical properties from the center to the end (e.g., edge) of the balloon to provide a more uniform current density in tissue along an axial length of the balloon.

In other embodiments, the expandable member is a self-expanding foam element and the energy conductor is a conductive fluid that can pass through the foam element. The foam element can be configured to contact a passageway with varying surface area, porosity (e.g., cell size), thickness, or physical properties to provide a more uniform current density in tissue along an axial length of the foam element. In still other examples, the expandable member can be a balloon, foam element, basket, array, mechanical scaffold or other item that expands, and the energy conductor can include a plurality of separate electrodes carried by the expandable member and different resistors coupled to the electrodes to control the current density in tissue proximate to the electrodes. In further embodiments, the expandable member may additionally comprise the energy conductor (e.g., metallic basket, electrode array, conductive foam, etc.) with different resistors coupled thereto to provide a more uniform current density in proximate tissue along a length thereof. For instance, foam may be coated or impregnated with carbon, silver, or other conductive filler, wherein a conductive filler concentration may be varied so as to achieve a uniform current density in the tissue.

Specific details of several embodiments of treatment devices, systems and methods for delivering energy to passageways in a patient are described below with respect to delivering radio frequency energy to airways in a lung of a patient to treat asthma. Other embodiments of the technology, however, can deliver other energy modalities to lung airways or other tissues (e.g., body cavities, skin, etc.) or passageways (e.g., blood vessel) for treating other indications. For example, the system can be configured to deliver thermal (resistive and/or infrared), microwave, ultrasonic (e.g., HIFU), cryo-ablation, or other types of energy modalities to tissue. Moreover, several other embodiments of the invention can have different configurations, components, or procedures than those described in this section. As such, several of the details set forth below are provided to describe the following examples in a manner sufficient to enable a person skilled in the relevant art to practice, make and use the described examples without undue experimentation. Several of the details and advantages described below, however, may not be necessary to practice certain embodiments of the technology. Additionally, the technology may include other embodiments and methods that are within the scope of the claims but are not described in detail. Moreover, the particular features, structures, routines, steps, or characteristics may be combined in any suitable manner in one or more embodiments of the technology.

### Embodiments of Treatment Systems

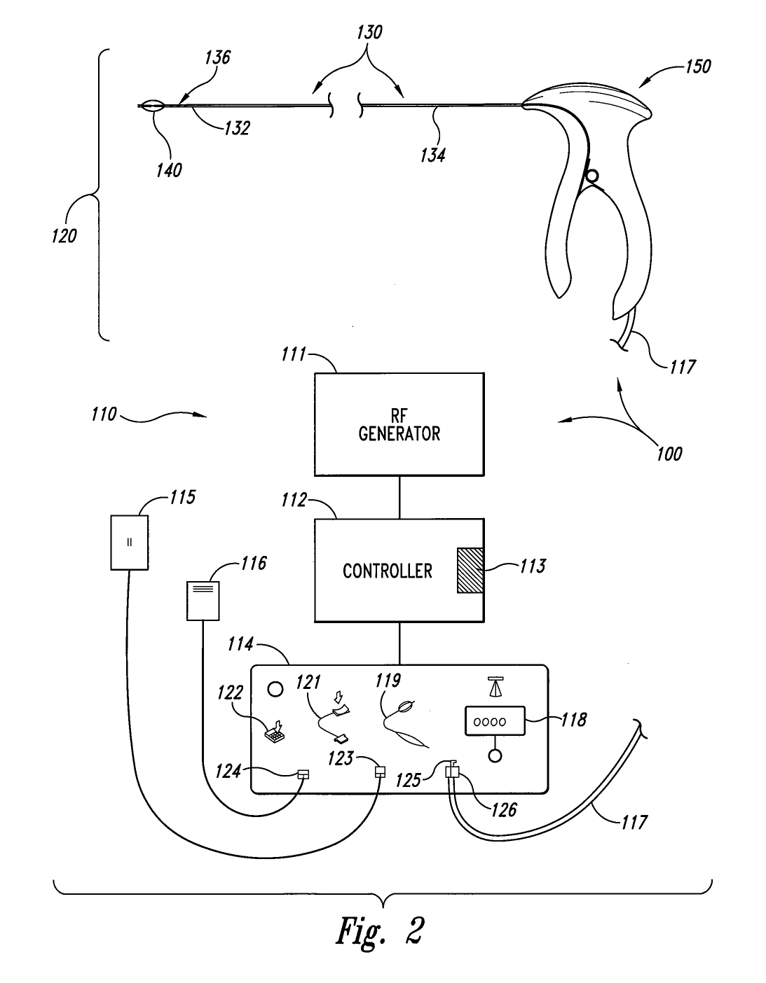

FIG. 2 is a schematic view illustrating a system 100 for delivering energy to passageways in a patient having a power/control unit 110 and an energy delivery device 120 in accordance with an embodiment of the technology. The power/control unit 110 can include an energy generator 111 (e.g., power supply), a controller 112 having a processor 113, and a user interface 114. The energy generator 111 and controller 112 can provide radio frequency (RF) energy to the energy delivery device 120, but in other embodiments the energy generator 111 and controller 112 can provide other energy modalities as explained above. The controller 112 can contain safety algorithms and other control algorithms that control (i) the power output to the energy delivery device 120 and (ii) the indicators 118, 119, 121, 122 of the user interface 114. The power/control unit 110 can further include one or more connections 123, 124, 125 for an optional return electrode 115 for monopolar RF configurations, an optional switch 116 (e.g., an actuation pedal) for directing the controller 112 to cause the energy generator 111 to provide energy, and a conductive line 117 and connector 126 coupled to the energy delivery device 120. It will be appreciated that the depictions herein are for illustrative purposes only and do not necessarily reflect the actual shape, size, or dimensions of the system or device.

The energy delivery device 120 is an example of a treatment device for treating asthma or other indications associated with passageways in a human. The embodiment of the energy delivery device 120 illustrated in FIG. 2 includes an elongated body 130 with a distal portion 132 and a proximal portion 134, an energy applicator 140 at the distal portion 132, and a handle 150 at the proximal portion 134. The length of the elongated body 130 should be sufficient to access the target tissue in airways of the lung or other passageways targeted for treatment. For example, the length of the elongated body 130 can be from approximately 0.5-8 feet to allow passage through a bronchoscope and reach targeted airways deep within the lungs. The elongated body 130 can also be configured to treat airways as small as 3 mm in diameter, but the elongated body 130 is not limited to treating airways of any particular size such that airways smaller or larger than 3 mm may be treated. Typically, the energy applicator 140 expands/contracts to variable sizes to treat airways between 1-15 mm.

Several embodiments of the elongated body 130 are flexible catheters configured to slide through an incision or working lumen of an access device (e.g., bronchoscope, endoscope, etc.) while the energy applicator 140 is in a low-profile configuration. The elongated body 130 can also include a plurality of markers 136 at the distal section 132 to position the energy applicator 140 relative to an access device or an anatomical location (not shown in FIG. 2). Specific embodiments of elongated bodies with markers suitable for use in the system 100 are described in U.S. patent application Ser. Nos. 11/551,639 and 11/777,225 and in U.S. Published Application No. US2007/0106292, all of which are incorporated herein by reference in their entirety.

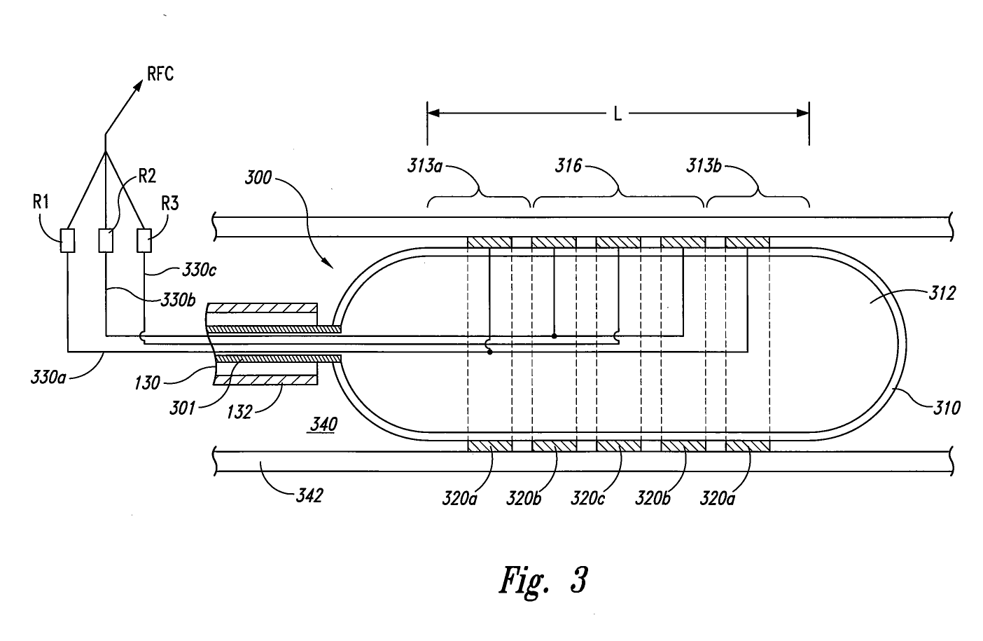

The energy applicator 140 can have an expandable member and at least one energy conductor configured to deliver energy to the tissue of an airway, passageway, or other body cavity in the patient. The expandable member, for example, can be configured to contact the passageway and present the energy conductor to the passageway. For example, the expandable member may comprise a balloon, foam element, basket, scaffold, array, or another type of member that is inflatable, self-expandable, and/or mechanically actuatable. The energy conductor can be a conductive fluid, a metal band, a metallic array, electrode segment, or other electrically conductive medium or element that can conduct a current in applications that deliver RF energy or other electrical energy. In other embodiments, the energy conductor can be an optic element for conducting an ultrasonic transmitter or other type of conductor suitable for the particular energy modality. Further details describing several embodiments of suitable energy applicators are described below with reference to FIGS. 3-6.

Referring back to FIG. 2, the illustrated example of the handle 150 is configured so that a single operator can hold an access device (e.g., a bronchoscope) in one hand (e.g., a first hand) and use the other hand (e.g., a second hand) to advance the elongated body 130 through a working lumen of the access device until the energy applicator 140 extends beyond the distal segment of the access device and is positioned at a desired target site. The handle 150 can also operate a pull wire or fluid valve that causes the expandable member and/or energy conductor to contact the sidewall of an airway passage while the catheter is held in place relative to the access device with the second hand. The same operator can also operate the switch 116 of the power/control unit 110 such that the entire procedure can be performed by a single person.

In several embodiments of the system, the controller 112 includes a processor that is generally configured to accept information from the system 100 and components associated with the system. The processor can process the information according to various algorithms to produce control signals for controlling the energy generator and/or produce information signals. The information signals produced by the processor may be directed to visual indicators, a digital display, or an audio tone generator of the user interface to inform the user of the system status, component status, procedure status, or any other useful information monitored by the system. The processor of the controller 112 may be a digital IC processor, analog processor, or any other suitable logic or control system that carries out the control algorithms.

Specific embodiments of systems that control power output to the energy delivery device are described in U.S. Pat. No. 7,104,987, U.S. Published Application No. US2006/0247746, and U.S. Provisional Patent Application No. 60/951,655, which are incorporated by reference herein in their entirety. The system 100 may deliver energy to target sites via the energy delivery device 120 in a variety of treatment patterns. Further details with respect to other designs and types of treatment devices, examples of energy, and/or examples of treatment patterns may be found in commonly-assigned U.S. Pat. No. 6,411,852.

### Embodiments of Energy Applicators and Energy Conductors

Several embodiments of the energy applicator 140 have a balloon, foam element, a basket, a scaffold, an array, or another type of expandable member and one or more energy conductors configured to contact target sites of a treatment area within a passageway. Several embodiments of the energy applicator have different resistances or other electrical properties (e.g., capacitance, inductance, impedance, etc.) to vary the amount of energy delivered to the tissue along the length of the applicator. In certain embodiments, the greatest resistances are at proximal and/or distal areas of the expandable member to compensate for edge effects.

FIG. 3 is a cross-sectional view illustrating an embodiment of an energy applicator 300 at the distal portion 132 of the elongated body 130. In this embodiment, the energy applicator 300 has a shaft 301, an expandable member defined by a non-conductive, non-porous balloon 310, and energy conductors defined by a plurality of ring/band electrodes 320a-c. The balloon 310 can be filled with a fluid 312 (e.g., liquid or gas) delivered via the shaft 301. The balloon 310 has a proximal area 313a, a distal area 313b, and a medial area 316 between the proximal and distal areas 313a-b. The electrodes 320a-c can be arranged such that first electrodes 320a are at the proximal and distal areas 313a-b, and at least one second electrode is at the medial area 316. The specific embodiment shown in FIG. 3 has two outer second electrodes 320b and a central second electrode 320c at the medial area 316. The electrodes 320a-c can be attached to an outer surface of the balloon 310 and coupled to conductive leads 330a-c, respectively, for connection to a radio frequency controller (RFC). The device 300 can also include resistors R1, R2, and R3 coupled to the electrodes 320a-c, respectively, along the energy deliver device 120 (e.g., at the handle 150). The resistances of the resistors R1-R3 can be configured such that R1>R2>R3. The energy applicator 300 accordingly provides a higher voltage across the tissue at the medial area 316 than at the proximal and distal areas 313a-b.

In operation, the power/control unit 110 (FIG. 2) provides power to the energy applicator 300 to deliver RF energy to a wall 342 of a passageway 340 via the electrodes 320a-c. Because of the different resistances (e.g., R1>R2>R3 divided by 2) associated with the electrodes 320a-c, the voltage provided to the first electrodes 320a at the proximal and distal areas 313a-b of the balloon 310 is less than the voltage provided to the outer second electrodes 320b, which is still less than the voltage provided to the central second electrode 320c. Thus, several embodiments of the energy applicator 300 control the energy delivered to the wall 342 along an axial length L of the passageway 340 by reducing a current at proximal and/or distal areas and/or increasing a current at a central area. The resistances R1-R3, for example, can be selected to provide a uniform or near uniform current density in the tissue of the wall 342 along the axial length L. This is illustrated in FIG. 3A, where the current density I in the tissue proximate to the entire length of the energy applicator 300, including edge E (e.g., 313a, 313b) and center C (e.g., 316) areas, is substantially uniform. It will be appreciated that the invention encompasses controlling electrical conductivity (e.g., resistance, capacitance, inductance, impedance, etc.) to more or less than three areas (i.e., proximal, distal, medial areas) or continuously varying electrical conductivity along a length. Further, it will be appreciated that other parameters (e.g., non-uniform electrode geometry, size, or material, filler concentration, etc.) may be utilized to vary the amount of energy (e.g., voltage, current) delivered to the tissue along the length of the applicator so that a substantially uniform current density may be achieved.

Another feature of several examples of the energy applicator 300 is that the ring electrodes 320a-c can also deliver energy around the circumference of the airway wall 342. The ring electrodes 320a-c can be continuous conductive bands as shown in FIG. 3, or they can be individual curved segments arranged around the circumference of the expandable member. Such discrete curved segments can be coupled to individual resistors to control the current density around the circumference of the airway wall as well. Thus, the energy applicator 300 may provide a uniform or near uniform current density in tissue around the circumference and/or along a length of a bronchial airway or other passageway. The length of an energy applicator may be greater than 5 mm and generally in a range from about 5 mm to about 30 mm. The current density in tissue along an axial length may generally be in a range from about 2 A/cm² to about 6 A/cm².

Several embodiments of energy applicators include a fluidic energy conductor and an expandable member through which the fluidic energy conductor can pass. The fluidic energy conductor may comprise a saline or water solution or other conductive liquid (e.g., other electrolytes). The expandable member, for example, can be a balloon or foam element permeable to water such that the conductive fluid can seep through the wall of the balloon or pass through the foam element to contact the wall of a treatment site within a passageway. In the case of a balloon, pores (e.g., micro-pores), holes, openings, apertures, orifices, and other conduits at the wall of the balloon may vary in size to vary the energy (e.g., voltage) delivered to the tissue along the length of the balloon. The conduits through the balloon can be configured to provide a uniform current density along a length of tissue. For example, the sizes (e.g., diameters), shapes, spacing and/or densities of pores can be greater at the medial area than the proximal and distal areas of the balloon to reduce or eliminate the edge effects and enable the energy delivery device to treat long treatment sites. Additionally, the fluid in the balloon can be cooled to protect a surface layer from thermal damage while still delivering the energy to deeper target tissue. For example, as the delivered energy heats or otherwise effects underlying smooth muscle tissue, the cooled liquid can control the temperature of an epithelial layer or other tissue layer to protect the surface of the airway wall or the passageway.

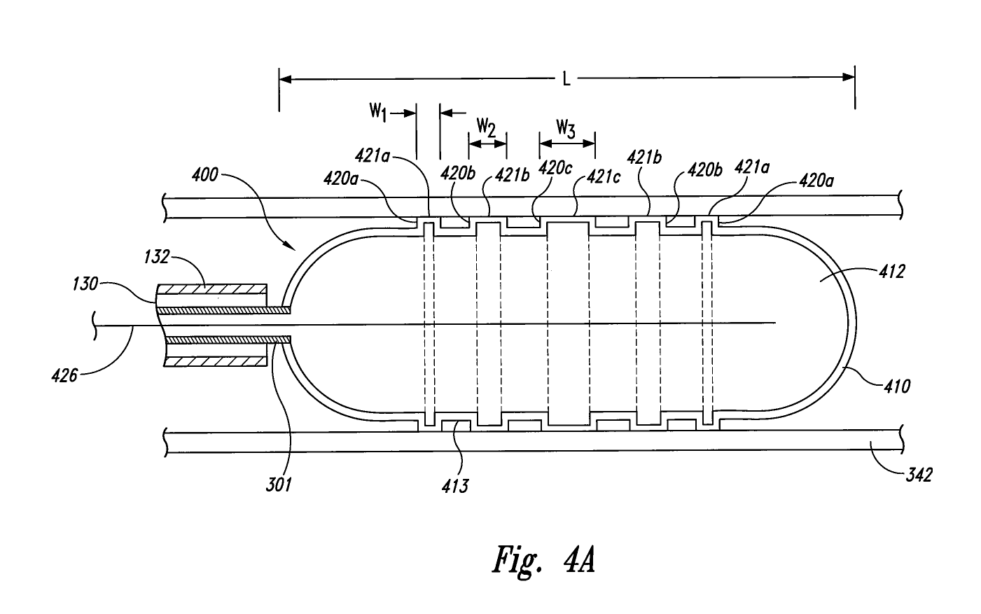

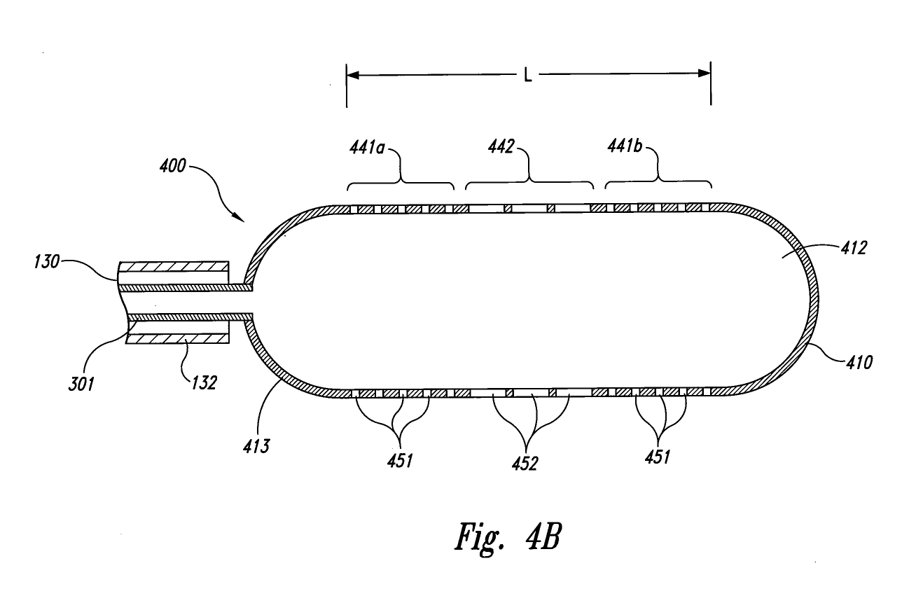

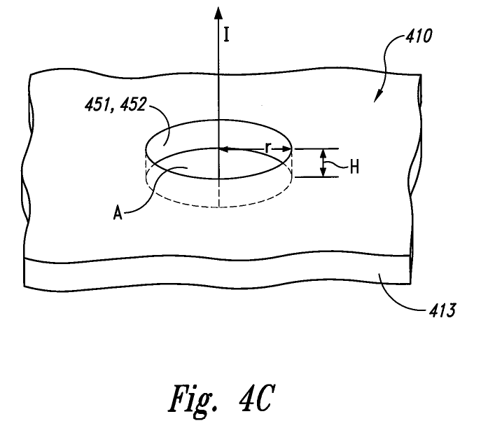

FIG. 4A is a cross-sectional view illustrating another embodiment of an energy applicator 400 in which the expandable member comprises a non-conductive balloon 410 and the energy conductor comprises a conductive liquid 412, such as a saline solution. The balloon 410 also includes conductive conduits 420a-c that vary in size (e.g., width) along an axial length L of the balloon 410. For example, first conduits 420a can have a width W₁ less than a width W₂ of outer second conduits 420b, which is less than a width W₃ of a central second conduit 420c. In one embodiment, the conduits 420a-c are cylindrical bands that project radially outwardly from a main portion 413 of the balloon 410 and have contact surfaces 421a-c, respectively, through which the conductive liquid 412 can carry an electrical current. The contact surfaces 421a-c, for example, can be a porous material or a sheet-like material having pores or holes through which the conductive solution can pass. The width of the contact surfaces 421a-c and/or the porosity or size of the holes of the contact surfaces 421a-c can be selected to provide a uniform or nearly uniform current density in tissue along the length L of the balloon 410.

The entire balloon 410 can comprise a material through which the conductive fluid can seep or otherwise pass. In other embodiments, the main portion 413 of the balloon and/or the sidewalls of the conduits can be an impermeable material, and only the contact surfaces 421a-c of the conduits 420a-c can be permeable to the conductive solution or ions of the conductive solution (e.g., a semipermeable cation or anion membrane). In particular embodiments, the balloon 410 may be made from suitable compliant materials such as urethane, silicone rubber, and the like or suitable non-compliant materials such as polyethylene terephthalate (PET), polytetrafluorethylene (PTFE), expanded PTFE (ePTFE), polyether block amide (PEBAX), and the like.

The energy applicator 400 can also have an electrode 426 in the balloon 410. The electrode 426 is connected to the power/control unit to deliver RF energy to the saline solution 412. Thus, as the saline solution seeps through the surface contact areas of the conduits 420a-c, the saline solution delivers RF energy to tissue of a passageway. The different sized conduits 420a-c and/or holes of the contact surfaces 421a-c can be configured to provide a uniform or near uniform current distribution in the tissue because of the different amount of energy delivered from the conduits 420a-c.

FIG. 4B illustrates another embodiment of the energy applicator 400. In this embodiment, the balloon 410 has a proximal area 441a, a distal area 441b, and a medial area 442 between the proximal and distal areas 441a-b. The energy applicator 400 further includes first pores 451 in the proximal and distal areas 441a-b, and second pores 452 in the medial area 442. The first and second pores 451-452 can be straight sided holes that pass through a sheet-like wall as shown, or the first and second pores 451-452 can be in first and second tortuously porous materials that have first and second porosities, respectively. The first pores 451 have first sizes and the second pores 452 have second sizes larger than the first sizes. As a result, the current applied at the medial area 452 is increased and/or the current applied at the proximal and distal areas 441a-b is reduced. The relative size and/or spacing of the first pores 451 and the second pores 452 can be selected to control the current density in the tissue of the wall of the passageway. In several embodiments, the sizes and/or spacing of the first pores 451 and the second pores 452 can be selected to provide at least a substantially uniform current distribution in the tissue.

FIG. 4C illustrates an embodiment of pores or holes, such as the pores 451-452, for use in the energy applicators 400 shown in FIGS. 4A and 4B in greater detail. In this embodiment, the pore has a height H (e.g., the thickness of the wall of the balloon) and a cross-sectional area A (e.g., equal to πr², where r is the pore radius), and a current I flows via the saline solution 412 through the pore. The resistance of the pore is directly proportional to the height H and inversely proportional to the area A and the conductivity σ of the saline. For example, the resistance R may be shown as:

R=L/σA,

and the current can be found by using Ohm's Law, V=IR. As a result, the proximal and distal areas 441 a-b with the smaller pores 451 has a higher resistance to the electrical current I than the medial area 442 with the larger pores 452. Similarly, increasing the thickness of the balloon wall H at the proximal areas 441a-b results in a higher resistance to the electrical current I than the medial area 442 with a smaller balloon wall thickness H. Suitable pore or hole diameters may be in a range from about 50 microns to about 150 microns and suitable wall thickness may be in a range from about 10 microns to about 100 microns.

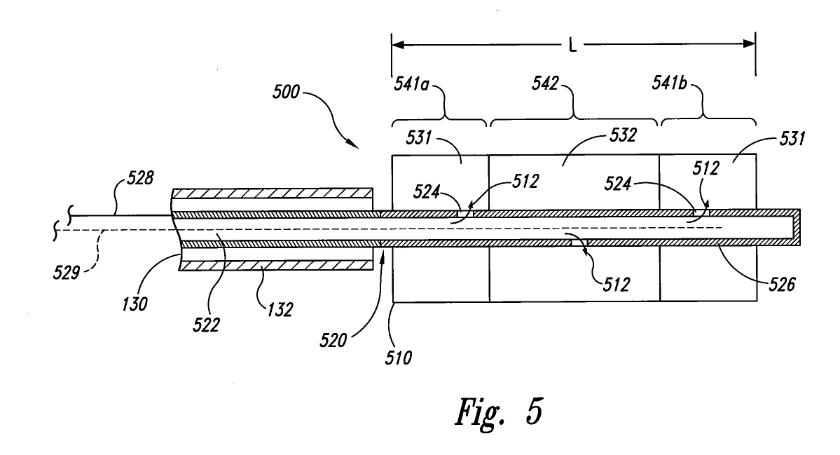

Alternate embodiments of expandable members of the energy applicators include a foam element through which a conductive liquid, such as a saline solution, can pass to the target tissue. The foam element may have different parameters that control the amount of energy delivered to the tissue at a target site. FIG. 5 is a side cross-sectional view illustrating an energy applicator 500 in which the expandable member is a self-expanding, non-conductive foam element 510 and the energy conductor is a conductive liquid 512, such as a saline solution, that can pass through the foam element. The energy applicator 500 can also include a shaft 520 (e.g., a catheter) having a fluid conduit 522 and a plurality of outlets 524. The conductive solution 512 can flow through the conduit 522 and the outlets 524 to the foam element 510. The shaft 520 can also have an emitter 526 (e.g., electrode) coupled to a conductive lead 528 that is connected to the power/control unit. In the embodiment shown in FIG. 5, the outlets 524 are holes in the wall of the emitter 526.

In operation, an operator moves the distal portion 132 of the elongated body 130 to a target site and deploys the energy applicator 500 to treat the tissue wall of a passageway. The energy applicator 500 can be deployed by pushing the shaft 520 distally out of the elongated body 130 or withdrawing the elongated body 130 proximally while holding the shaft 520. The elongated body 130 and/or the foam element 510 may be chamfered or tapered at proximal or distal ends to help facilitate deployment or retraction. The foam element 510 self expands to contact the passageway, and the conductive fluid 512 flows through the outlets 524 into the foam element 510. The conductive fluid 512 fills the interstitial spaces in the foam element 510 such that conductive fluid at the surface of the foam element 510 contacts the wall of an airway. The power/control unit transmits RF energy to the conductive fluid 512 via lead 528 and emitter 526. The emitter 526 may have a large contact surface to avoid the build up of high current densities which might cause vaporization of the fluid 512 through which current is being passed. In an alternative embodiment, the energy applicator can have an electrode 529 (shown in broken line) extending into the foam element 510 instead of or in addition to the lead 528 and emitter 526. In addition to energy delivery, the self expanding foam applicator may also facilitate mucus clearing in the airway prior to energy delivery to ensure against mucus heating and/or from the access scope for improved visualization.

The foam element 510 may have a variable porosity along the axial length L. In one example, the porosity gradually decreases from the middle of the length L to the proximal and distal ends of the foam element 510. The variable porosity controls the amount of saline solution that contacts the tissue, and thus the current density, at different regions along the length L to provide a uniform or nearly uniform current density in tissue at the target site. In alternative embodiments, the foam element 510 may include discrete sections having different porosities. For example, the foam element 510 can have first foam segments 531 at proximal and distal areas 541a-b and a second foam segment 532 at a medial area 542. The porosity of the second foam segment 532 can be greater than the first foam segments 531 to provide more energy to tissue at the medial area 542. The individual foam segments 531 and 532 may have different porosities based on the number, size and spacing of pores. The number of pores or other porosity parameters may accordingly be adjusted to control the current density in tissue along the length of the foam element 510.

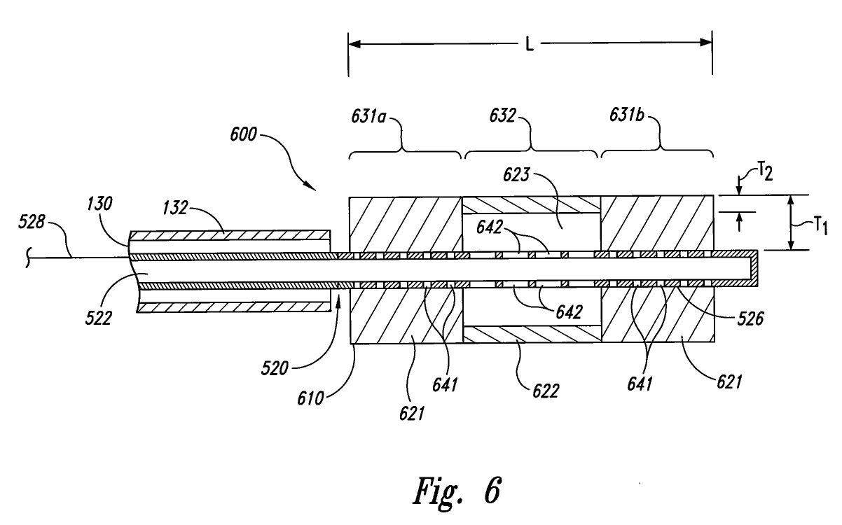

In other embodiments, the thickness at different regions of a foam element may differ to control the current density along the foam element. FIG. 6 is a cross-sectional view of an embodiment of an energy applicator 600 having a foam element 610 with multiple foam segments that have different thicknesses. The foam element 610, for example, can have first foam segments 621 with a first thickness T₁ at proximal and distal areas 631a-b and a second foam segment 622 with a second thickness T₂ at a medial area 632. The first thickness T₁ of the first foam segments 621 can be thicker than the second thickness T₂ of the second foam segment 622. The energy applicator 600 can accordingly have a cavity 623 in the second foam segment 622. The second foam segment 622 is accordingly less resistant to a flow of saline solution such that more saline seeps through the second foam segment 622 to the wall of the airway. Additionally, the density or porosity of the foam segments 621 and 622 may be different from each other as well.

The energy applicators 500 and 600 can also have a different configuration of outlets to control the flow of conductive solution to different segments of the foam elements. Referring to FIG. 6, for example, the emitter 526 can have first outlets 641 in the proximal and distal areas 631a-b and second outlets 642 in the medial area 632. The first outlets 641 are smaller than the second outlet 642 in the illustrated example to provide a higher flow rate of conductive solution to the second foam segment 622 than the first foam segments 621. In other embodiments, however, the porosity, thickness, fluid pressurization, and/or outlet configuration can be different. Suitable materials for the foam elements 510, 610 include shape memory material, silicone open cell foam, PORON microcellular urethane foam, cellulose fiber, hydrophilic sponge material (e.g., elastomeric urethane), and the like.

The energy applicators 140, 300, 400, 500 and 600 described above can provide monopolar “electrodes” with controlled electrical properties relative to the length and/or circumference of the applicators. Several embodiments of the systems and methods can accordingly have large electrically conductive contact areas to treat large areas of tissue during a single treatment cycle without extensive edge effects that would otherwise occur with such large electrical contact areas. As such, several embodiments of the system may reduce the time needed for treating tissue within bronchial airways or other body cavities. This enables a facility to treat more patients and enhances the experience and convenience for the patients. The energy applicators 300, 400, 500, 600 described herein may also be used in a manner that protects the epithelium of the tissue while controlling the tissue depth and/or penetration of the energy delivery of the device. For example, as described above, the inflation medium or conductive solution can be cooled to maintain the temperature of the tissue wall of the passageway in a manner that protects the epithelial layer. Cooling may be applied before, during, and/or after energy delivery.

### CONCLUSION

The headings provided herein are for convenience only and are not intended to limit or interpret the scope or meaning of the claimed technology. Additionally, unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. When the claims use the word “or” in reference to a list of two or more items, that word covers all of the following interpretations of the word: any of the items in the list, all of the items in the list, and any combination of the items in the list.

The foregoing specific embodiments of the invention have been described for the purposes of illustration, but various modifications may be made without deviating from the scope of the invention. For example, specific features or processes of the various examples described above can be combined to provide further examples. Aspects of the technology may accordingly be modified, if necessary, to employ treatment devices with a plurality of treatment units, thermally conductive devices with various configurations, and concepts of the various patents, applications, and publications incorporated by reference to provide yet further embodiments of the technology. These and other changes, therefore, can be made to the technology in light of the above-detailed description. In general, in the following claims, the terms used should not be construed to limit the technology to the specific examples disclosed in the specification and the claims, but should be construed to include all embodiments in accordance with the claims. Accordingly, the technology is not limited by the disclosure, but instead its scope is to be determined entirely by the following claims.

We claim:

1. An energy delivery device for use in a body conduit or cavity, the device comprising:
an elongated body having a proximal portion and a distal portion; and
an energy applicator at the distal portion of the elongated body, wherein the energy applicator has a proximal area, a distal area, and a medial area between the proximal and distal areas, and wherein the energy applicator is configured to deliver a first voltage at the proximal and distal areas and a second voltage greater than the first voltage at the medial area.

2. The device of claim 1, wherein the energy applicator further comprises an expandable member.

3. The device of claim 2, wherein the expandable member comprises a balloon through which a conductive fluid can pass, and wherein the proximal and distal areas have a first physical property configured to create a first resistance and the medial area has a second physical property configured to create a second resistance less than the first resistance.

4. The device of claim 2, wherein the expandable member comprises a balloon through which a conductive fluid can pass, and wherein the balloon has a first porosity at the proximal and distal areas and a second porosity greater than the first porosity at the medial area such that resistance is greater in the proximal and distal areas than in the medial area.

5. The device of claim 4, wherein the proximal and distal areas have first pores and the medial area has second pores larger than the first pores.

6. The device of claim 2, wherein the expandable member comprises a balloon through which a conductive fluid can pass, and wherein the balloon has a first wall thickness at the proximal and distal areas and the medial area has a second wall thickness less than the first wall thickness.

7. The device of claim 2, wherein the expandable member comprises a self-expanding foam element through which a conductive fluid can pass, and wherein the proximal and distal areas have a first physical property configured to create a first resistance and the medial area has a second physical property configured to create a second resistance less than the first resistance.

8. The device of claim 2, wherein the expandable member comprises a self-expanding foam element through which a conductive fluid can pass, and wherein the foam element has first foam segments with a first porosity at the proximal and distal areas and a second foam segment at the medial area with a second porosity greater than the first porosity such that resistance is greater at the proximal and distal areas than the medial area.

9. The device of claim 8, wherein the proximal and distal areas have first pores and the medial area has second pores larger than the first pores.

10. The device of claim 2, wherein the expandable member comprises a self-expanding foam element through which a conductive fluid can pass, and wherein the foam element has first foam segments with a first thickness at the proximal and distal areas and a second foam segment at the medial area with a second thickness less than the first thickness.

11. The device of claim 2, wherein the expandable member comprises a balloon or foam element through which a conductive fluid can pass and the energy applicator further comprises a conductive emitter within the expandable member having first outlets in the proximal and distal areas and second outlets in the medial area, and wherein the first and second outlets are configured to provide different fluid flow characteristics through the balloon or foam element in the medial area compared to the proximal and distal areas.

12. The device of claim 2, wherein the energy applicator further comprises an energy conductor at the expandable member.

13. The device of claim 12, wherein the energy conductor comprises a first electrode at the proximal and distal areas, a first resistor having a first resistance coupled to the first electrodes, a second electrode at the medial area, and a second resistor having a second resistance less than the first resistance coupled to the second electrode.

14. The device of claim 2, wherein the expandable member comprises a conductive foam element or metallic array having a first resistance at the proximal and distal areas and a second resistance less than the first resistance at the medial area.

15. An energy delivery device for use in a body conduit or cavity, the device comprising:
an elongated body having a proximal portion and a distal portion;
an expandable member at the distal portion of the elongated body; and
an energy conductor at the expandable member, wherein the expandable member and the energy conductor are configured to vary an electrical conductivity along an axial length of the energy conductor so as to achieve a substantially uniform current density along a length of tissue.

16. The device of claim 15, wherein the expandable member and the energy conductor are configured to provide a first voltage at proximal and distal areas of the expandable member and a second voltage greater than the first voltage at a medial area of the expandable member.

17. The device of claim 15, wherein the energy conductor comprises first conductive segments at proximal and distal areas of the expandable member, a second conductive segment at a medial area of the expandable member, first resistors coupled to the first conductive segments, and a second resistor coupled to the second conductive segment, wherein the first resistors have a higher resistance than the second resistor.

18. The device of claim 15, wherein the energy conductor comprises a conductive fluid and the expandable member comprises one of a non-conductive balloon or foam element through which the conductive fluid can pass, wherein the one of the balloon or foam element has proximal and distal areas and a medial area between the proximal and distal areas, and wherein the medial area is configured to deliver a different amount of energy than the proximal and distal areas.

19. The device of claim 18, wherein the one of the balloon or foam element has first pores with first diameters at the proximal and distal areas and second pores with second diameters greater than the first diameters at the medial area.

20. The device of claim 18, wherein the one of the balloon or foam element has a first thickness at the proximal and distal areas and a second thickness at the medial area, and wherein the first thickness is greater than the second thickness.

21. The device of claim 18, further comprising a fluid conduit connected to the one of the balloon or foam element for the delivery of the conductive fluid.

22. A method for treating an internal airway in a lung of a patient, the method comprising:
positioning an elongated body of a treatment device in a lung airway of a patient;
expanding an energy applicator at a distal portion of the elongated body to contact a wall of the lung airway; and
delivering energy to the wall of the airway by varying an electrical conductivity along an axial length of the energy applicator so as to reduce edge effects along a length of target tissue.

23. The method of claim 22, wherein delivering comprises applying a first voltage to proximal and distal areas of the energy applicator and a second voltage greater than the first voltage to a medial area of the energy applicator.

24. The method of claim 22, wherein delivering comprises passing a conductive fluid through the energy applicator.

25. The method of claim 22, wherein delivering comprising applying current in a monopolar fashion.

26. The method of claim 22, wherein delivering comprises circumferentially and longitudinally heating tissue.

27. The method of claim 22, wherein expanding comprises inflating, self-expanding, or mechanically actuating the energy applicator.

28. The method of claim 22, wherein the target tissue comprises smooth muscle tissue.

29. The method of claim 22, further comprising cooling a tissue layer adjacent the target tissue.

30. The method of claim 29, wherein the adjacent layer comprises epithelial tissue.
